(19) United States
(12) Patent Application Publication
(10) Pub. No.: US 2024/0016993 A1
(43) Pub. Date: Jan. 18, 2024

(54) VACUUM DRAINAGE MANIFOLD SYSTEMS AND RELATED METHODS

(71) Applicant: Merit Medical Systems, Inc., South Jordan, UT (US)

(72) Inventors: Michael Dean Haslam, Sandy, UT (US); Alex Singleton, Sandy, UT (US); Diana Laree Hardy, Sandy, UT (US); Jim Mottola, West Jordan, UT (US)

(21) Appl. No.: 18/349,545

(22) Filed: Jul. 10, 2023

Related U.S. Application Data
(60) Provisional application No. 63/368,213, filed on Jul. 12, 2022.

(51) Int. Cl. A61M 1/00; A61M 39/22; A61M 39/20

(52) U.S. Cl. CPC A61M 1/64; A61M 1/84; A61M 39/22; A61M 39/20; A61M 2039/229

(57) ABSTRACT

Devices, systems, kits, and methods used to collect drainage fluid from a patient are disclosed. The systems or kits include a multi-port manifold, and one or more drainage tubes to fluidly connect vacuum drainage bottles with the multi-port manifold. One or more vacuum drainage bottles can also be included in the kit as desired. The method of drainage fluid collection can include filling two vacuum drainage bottles simultaneously with drainage fluid, then filling a third vacuum drainage bottle with drainage fluid, and then filling a fourth vacuum drainage bottle with drainage fluid.

### RELATED APPLICATIONS

This application claims priority to U.S. Provisional Application No. 63/368,213, filed Jul. 12, 2022, titled VACUUM DRAINAGE MANIFOLD SYSTEMS AND RELATED METHODS, which is incorporated herein by reference in its entirety.

### TECHNICAL FIELD

The present disclosure relates generally to drainage devices used to drain fluids from a patient. More specifically, the present disclosure relates to a vacuum bottle drainage system or kit.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1A is a side view of an embodiment of a vacuum drainage collection system or kit.

FIG. 1B is a perspective view of a cap of a vacuum drainage bottle of the vacuum drainage collection system of FIG. 1A.

FIG. 2A is a side view of the vacuum drainage collection system of FIG. 1A in a set-up state.

FIG. 2B is a top view of valve handles of the vacuum drainage collection system of FIG. 2A in the set-up state.

FIG. 3A is a side view of the vacuum drainage collection system of FIG. 1A in a first drainage state.

FIG. 3B is a top view of the valve handles of the vacuum drainage collection system of FIG. 3A in the first drainage state.

FIG. 4A is a side view of the vacuum drainage collection system of FIG. 1A in a second drainage state.

FIG. 4B is a top view of valve handles of the vacuum drainage collection system of FIG. 4A in the second drainage state.

FIG. 5A is a side view of the vacuum drainage collection system of FIG. 1A in a third drainage state.

FIG. 5B is a top view of valve handles of the vacuum drainage collection system of FIG. 5A in the third drainage state.

FIG. 6A is a side view of the vacuum drainage collection system of FIG. 1A in a drainage fluid sampling state.

FIG. 6B is a top view of valve handles of the vacuum drainage collection system of FIG. 6A in the drainage fluid sampling state.

FIG. 7 is a side view of an embodiment of a vacuum drainage bottle having a thermochromic material.

FIG. 8 is a side view of an embodiment of a vacuum drainage bottle having a thermochromic label.

### DETAILED DESCRIPTION

In some instances, body fluids may need to be withdrawn from a patient in the course of medical treatment. For example, two medical procedures requiring fluid removal are thoracentesis and paracentesis. In paracentesis, peritoneal fluid is aspirated from the abdomen. In thoracentesis, pleural fluid is aspirated from the thoracic cavity. In certain instances, paracentesis and thoracentesis have been observed to provide quick and effective relief with few adverse side effects. Relatively large volumes of fluid, such as four liters, may be withdrawn from a patient during a single procedure. The paracentesis and thoracentesis procedures can be conducted in a health care facility by health care workers or in a patient's home by the patient. Many existing devices are capable of performing paracentesis and thoracentesis. At its simplest, a paracentesis or thoracentesis device need only include a hollow needle with one end inserted into the patient and the other end attached to a negative gauge pressure or vacuum device, such as a vacuum drainage collection bottle. As used herein, a vacuum can refer to a negative gauge pressure or a pressure that is less than ambient or atmospheric pressure.

A vacuum drainage collection system or kit of the present disclosure can include a multi-port manifold, one or more drainage tubes couplable to one or more vacuum drainage bottles and one or more ports of the multi-port manifold, and a stopcock valve couplable to a port of the multi-port manifold. The vacuum drainage collection system or kit may further include a drainage fluid sampling device. The vacuum drainage collection system or kit may further include an instructions for use (IFU) sheet or directions for accessing instructions (e.g., a quick response (QR) code) on how to use the vacuum drainage collection system or kit to drain fluid from a patient's body. One or more vacuum drainage bottles can also be included in the vacuum drainage collection system or kit as desired.

In another embodiment, a vacuum drainage collection system or kit of the present disclosure can include one or more vacuum drainage bottles having a cap configured to selectively open and close the vacuum drainage bottle, a multi-port manifold, one or more drainage tubes couplable to the one or more vacuum drainage bottles and one or more ports of the multi-port manifold, and a stopcock valve couplable to a port of the multi-port manifold. The disclosed vacuum drainage collection system or kit may further include an instructions for use (IFU) sheet or directions for accessing instructions (e.g., a quick response (QR) code) on how to use the vacuum drainage collection system or kit to drain fluid from a patient's body.

A method of using the vacuum drainage collection system or kit to drain fluid from a patient's body within the scope of this disclosure can include coupling a first drainage tube to a first vacuum drainage bottle and a first port of the multi-port manifold; coupling a second drainage tube to a second vacuum drainage bottle and a second port of the multi-port manifold; opening a valve of the first vacuum drainage bottle to expose the first drainage tube to a vacuum; opening a valve of the second vacuum drainage bottle to expose the second drainage tube to a vacuum; opening the second port and the first port of the multi-port manifold to allow drainage fluid to flow through the first and second drainage tubes and into the first and second vacuum drainage bottles; and filling the first and second vacuum drainage bottles with drainage fluid simultaneously.

In some embodiments, the method of use may further include coupling a third drainage tube to a third vacuum drainage bottle and a third port of the multi-port manifold; opening the third vacuum drainage bottle to expose the third drainage tube to a vacuum; opening the third port to allow drainage fluid to flow through the third drainage tube and into the third vacuum drainage bottle; and filling the third vacuum drainage bottle with drainage fluid.

In additional embodiments, the method of use may further include coupling a fourth drainage tube to a fourth vacuum drainage bottle and a fourth port of the multi-port manifold; opening the fourth vacuum drainage bottle to expose the fourth drainage tube to a vacuum; opening the fourth port to allow drainage fluid to flow through the fourth drainage tube and into the fourth vacuum drainage bottle; and filling the fourth vacuum drainage bottle with drainage fluid. Additional vacuum drainage bottles can be used as desired.

In certain embodiments, the method of use may further include coupling a drainage fluid sampling device to a stopcock valve which is coupled to another port (e.g., a fifth port) of the multi-port manifold; opening the stopcock valve; and filling the drainage fluid sampling device with drainage fluid.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure but is merely representative of various embodiments. While the various aspects of the embodiments are presented in drawings, the drawings are not necessarily drawn to scale unless specifically indicated.

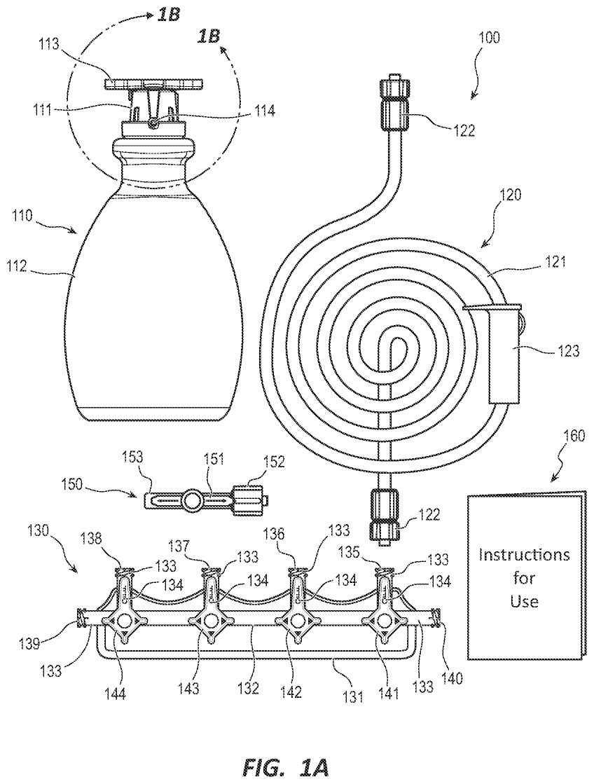

FIGS. 1A-8 illustrate different views of various embodiments of a vacuum drainage collection system or kit and related components. In certain views the vacuum drainage collection system or kit may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

As illustrated in FIG. 1A, an embodiment of a vacuum drainage collection system or kit 100 can include a vacuum drainage bottle 110, a drainage tube 120, a multi-port manifold 130, a stopcock valve 150, and an optional IFU sheet 160. In other embodiments, the vacuum drainage collection system or kit 100 may include two, three, four, or more vacuum drainage bottles 110 and two, three, four, five, or more drainage tubes 120. In yet other embodiments, one or more components can be separate from the vacuum drainage collection system or kit 100 and added to the vacuum drainage collection system or kit 100 during use. For instance, one or more vacuum drainage bottles 110 can be obtained and used in conjunction with a vacuum drainage collection system or kit 100 that includes a multi-port manifold 130, a stopcock valve 150, and an optional IFU sheet 160.

As illustrated in FIGS. 1A and 1B, the vacuum drainage bottle 110 may include a cap 111 coupled to a container 112. Other types of vacuum drainage bottles can also be used, such as those that include different types of caps or seals. The cap 111 can include a rotatable valve handle 113 and an inlet port 114. As illustrated in FIG. 1B, the valve handle 113 may include one or more arms 116 extending radially outward from a central portion of the valve handle 113. A position indicium 115 may be disposed on one of the arms 116 to indicate whether the valve handle 113 is in a closed state or an open state. In the illustrated embodiment of FIG. 1B, the indicium 115 includes and arrowhead shape that is pointed away from the inlet port 114 indicating that the valve handle 113 is in the closed state. When the valve handle 113 is rotated such that the indicium 115 is in alignment with the inlet port 114, the indicium 115 may indicate that the valve handle 113 is in the open state. In other embodiments, the valve handle 113 may include any suitable shape, such as a round knob, an oval knob, etc. and the position indicium 115 may include any suitable type, such as a line, a circle, a color, etc.

The vacuum drainage bottle 110 may be of any suitable form capable of retaining an internal vacuum for an extended length of time and to retain drainage fluid for transport and disposal. For example, as illustrated in FIG. 1A, the vacuum drainage bottle 110 includes a truncated prolate spheroid shape or truncated football shape with a broad closed base. A volume of the vacuum drainage bottle 110 may range from about 250 milliliters to about 1,250 milliliters and may be about 1,000 milliliters. The cap 111 can be sealingly coupled to an open end of the vacuum drainage bottle 110. The vacuum drainage bottle 110 can be formed of a transparent or translucent material to permit observation of the volume of the drainage fluid within the vacuum drainage bottle 110. For example, the material may be one or more of glass and polymeric materials, such as polycarbonate, polyesters, cyclic olefin copolymers, etc.

As illustrated in FIG. 1A, the drainage tube 120 can include a flexible tubing 121 with a connector 122 coupled to each end of the tubing 121. In the illustrated embodiment of FIG. 1A, the connectors 122 are male Luer lock fittings configured to couple with a female Luer lock fitting of the inlet port 114 and a female Luer lock fitting of a port of the multi-port manifold 130. The tubing 121 includes a bore extending throughout the length of the tubing 121. The bore can be in fluid communication with the vacuum drainage bottle 110 and the multi-port manifold 130 to allow drainage fluid to flow from the multi-port manifold 130 to the vacuum drainage bottle 110, as will be described below. The tubing 121 may be formed of any suitable flexible transparent or translucent material. For example, the tubing 121 may be formed from polyvinyl chloride, polyethylene, thermoplastic elastomer, silicone, etc. Other materials are contemplated. The drainage tube 120 can optionally include an adjustable clamp 123 configured to adjustably control a flow rate of drainage fluid flowing through the tubing 121. As depicted in the embodiment of FIG. 1A, the adjustable clamp 123 may be a roller clamp. In other embodiments, the adjustable clamp 123 can be any suitable adjustable clamp, such as a slide clamp, a pinch clamp, etc. In certain embodiments, the tube 120 may include a one-way valve to prevent backflow of drainage fluid from the vacuum drainage bottle 110 (e.g., such as backflow that could be caused by the opening another vacuum drainage bottle).

The tubing 121 can be various lengths. In some embodiments, the vacuum drainage collection system or kit 100 includes a plurality of drainage tubes 120 having different lengths. For instance, the vacuum drainage collection system or kit 100 can include a plurality of drainage tubes 120 (e.g., two, three, four, five, or more) having shorter lengths for coupling one or more vacuum drainage bottles 110 to the multi-port manifold 130, and at least one drainage tube 120 having a longer length for coupling the multi-port manifold 130 to a drainage catheter inserted into a patient's body.

As illustrated in FIG. 1A, the multi-port manifold 130 can include a body 131, an enclosed fluid channel 132 extending longitudinally through the body 131, and ports 133 extending from the body 131 and in fluid communication with the fluid channel 132. One or more of the ports 133 can include a rotatable valve 134 (e.g., stopcock valve) configured to allow or prevent flow of drainage fluid through the fluid channel 132 and/or the ports 133. Each of the plurality of ports 133 can include a female Luer lock fitting. In the depicted embodiment of FIG. 1A, the multi-port manifold 130 includes a first side port 135 including a first rotatable valve 141, a second side port 136 including a second rotatable valve 142, a third side port 137 including a third rotatable valve 143, a fourth side port 138 including a fourth rotatable valve 144, a fifth end port 139, and a sixth end port 140. Each of the rotatable valves 141, 142, 143, 144 can be individually operable to control fluid flow through the fluid channel 132 and/or the side ports 135, 136, 137, 138, respectively.

As illustrated in FIG. 1A, the stopcock valve 150 can include a rotatable handle 151, a male Luer lock fitting 152 disposed at a first end, and a female Luer fitting 153 disposed at a second end opposite the first end. A bore can extend through the stopcock valve 150 from the first end to the second end. The male Luer lock fitting 152 may be configured to couple with a port 133 of the multi-port manifold 130 to fluidly couple the bore with the fluid channel 132 of the multi-port manifold 130. The female Luer fitting 153 can be configured to couple with a drainage fluid sampling device to sample drainage fluid from the multi-port manifold 130, as will be described below. The rotatable handle 151 can be selectively rotated to a first position to prevent flow of drainage fluid through the bore and to a second position to allow flow of drainage fluid through the bore and into the drainage fluid sampling device. The stopcock valve can also include other types of fittings as desired (e.g., the stopcock valve can include male Luer lock fittings on both ends, etc.).

As illustrated in FIG. 1A, the IFU sheet 160 can be a printed sheet or booklet comprising printed text and/or illustrations of step-by-step instructions, warnings, cautions, and other information regarding use of the vacuum drainage collection system or kit 100 to perform a procedure to drain fluid from the patient's body. For example, the IFU sheet 160 may provide step-by-step instructions of how to perform a paracentesis and/or thoracentesis procedure. In other embodiments, the vacuum drainage collection system or kit 100 can include directions for accessing the instructions or IFU sheet, such as from a website. For instances, the vacuum drainage collection system or kit 100 can include a QR code printed on a surface (e.g., a label). The QR code may be read by a personal digital assistant (PDA) (e.g., a smart phone, a tablet). The PDA may obtain the instructions from a website and digitally display the text and/or illustrations of the step-by-step instructions, warnings, cautions, and other information regarding use of the vacuum drainage collection system or kit 100. Other types of directions for accessing the instructions are also contemplated.

The vacuum drainage collection system or kit 100 may be packaged in packaging suitable to protect the components from damage during shipping and to maintain sterility of the components for an extended period time while the vacuum drainage collection system or kit 100 sits on a shelf of a healthcare facility or patient's home. For example, the components of the vacuum drainage collection system or kit 100 may be disposed in a thermoformed tray including cavities configured to receive each of the components. The tray may then be disposed in an outer bag and the bag sealed. In another embodiment, the thermoformed tray may be sealed with a lid.

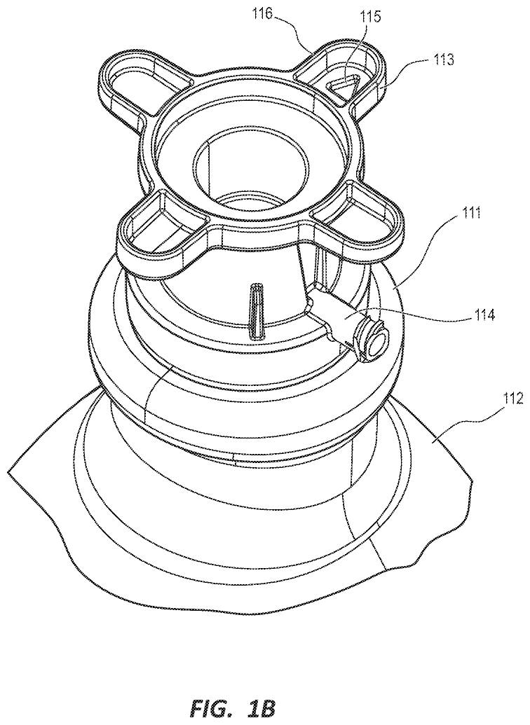

FIGS. 2A-6B illustrate a method of using the vacuum drainage collection system or kit 100. FIGS. 2A and 2B illustrate the vacuum drainage collection system or kit 100 in a set-up state. During use, and as illustrated in FIGS. 2A and 2B, a first drainage tube 120a is coupled to a first vacuum drainage bottle 110a and to the first port 135 of the multi-port manifold 130. A first valve handle 113a is positioned in a closed position. The first rotatable valve 141 is positioned such that drainage fluid is prevented from flowing through the fluid channel 132 and the first port 135. A second drainage tube 120b is coupled to a second vacuum drainage bottle 110b and to the second port 136 of the multi-port manifold 130. A second valve handle 113b is positioned in a closed position. The second rotatable valve 142 is positioned such that drainage fluid is allowed to flow through the fluid channel 132 and the second port 136. A third drainage tube 120c is coupled to a third vacuum drainage bottle 110c and to the third port 137 of the multi-port manifold 130. A third valve handle 113c is positioned in a closed position. The third rotatable valve 143 is positioned such that drainage fluid is allowed to flow through the fluid channel 132 and prevented from flowing through the third port 137. A fourth drainage tube 120d is coupled to a fourth vacuum drainage bottle 110d and to the fourth port 138 of the multi-port manifold 130. A fourth valve handle 113d is positioned in a closed position. The fourth rotatable valve 144 is positioned such that drainage fluid is allowed to flow through the fluid channel 132 and prevented from flowing through the fourth port 138.

With continued reference to FIGS. 2A and 2B, the stopcock valve 150 is coupled to the fifth port 139 of the multi-port manifold 130. The rotatable handle 151 is positioned such that drainage fluid is prevented from flowing through the stopcock valve 150. A fifth drainage tube 120e is coupled to the sixth port 140 of the multi-port manifold 130 and to a drainage catheter (not shown) inserted into a patient's body. As can be appreciated, while the illustrated embodiment depicts four vacuum drainage bottles (100a, 100b, 100c, 100d), fewer vacuum drainage bottles could also be used with the multi-port manifold 130, such as one, two, or three vacuum drainage bottles. Additionally, in other embodiments, the multi-port manifold 130 can be configured for use with more vacuum drainage bottles, such as five or more.

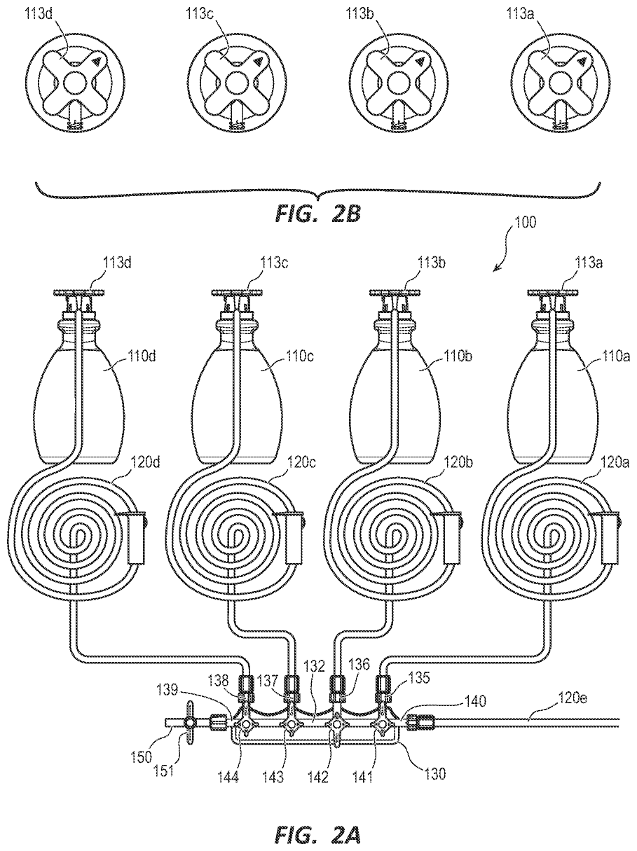

FIGS. 3A and 3B illustrate the vacuum drainage collection system or kit 100 in a first drainage state where the first vacuum drainage bottle 110a and the second vacuum drainage bottle 110b are at least partially filled with drainage fluid simultaneously. As illustrated, the first valve handle 113a is rotated to an open position to expose the first drainage tube 120a to the vacuum of the first vacuum drainage bottle 110a. The second valve handle 113b is rotated to an open position to expose the second drainage tube 120b to the vacuum of the second vacuum drainage bottle 110b. With the second rotatable valve 142 in the open position, the first rotatable valve 141 can be rotated to a position to expose the fifth drainage tube 120e to the vacuum of the first vacuum drainage bottle 110a and the second vacuum drainage bottle 110b to allow flow of the drainage fluid 102 under the vacuum through the fifth drainage tube 120e, the fluid channel 132, the first and second side ports 135, 136, the first and second drainage tubes 120a, 120b, and into the first and second vacuum drainage bottles 110a, 110b simultaneously. Alternatively, in another embodiment, the drainage bottles 110a, 110b can be used to collect drainage fluid sequentially (e.g., into the first drainage bottle 110a, and then into the second drainage bottle 110b via use of the multi-port manifold).

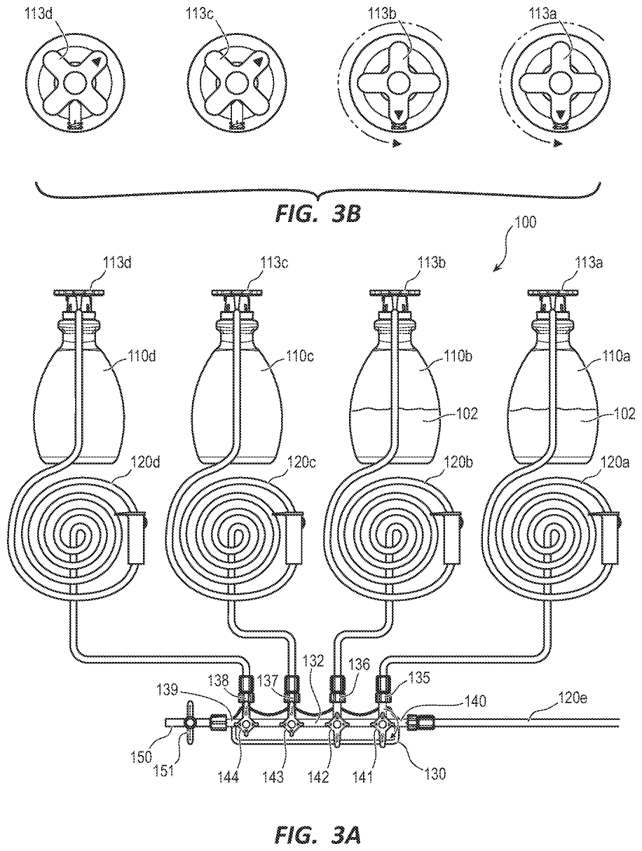

As illustrated in FIGS. 4A and 4B, when the first vacuum drainage bottle 110a and the second vacuum drainage bottle 110b are filled with a drainage fluid 102 to desirable levels, the third valve handle 113c is rotated to the open state to expose the third drainage tube 120c to the vacuum of the third vacuum drainage bottle 110c. The third rotatable valve 143 is rotated to expose the fluid channel 132 and the fifth drainage tube 120e to the vacuum of the third vacuum drainage bottle 110c. The drainage fluid 102 is drawn through the fifth drainage tube 120e, the fluid channel 132, the third side port 137, the third drainage tube 120c, and into the third vacuum drainage bottle 110c. In some embodiments, when the third valve handle 113c is rotated to the open state, the drainage fluid 102 level within one or more of the first and second vacuum drainage bottles 110a, 110b can increase due to the exposure of one or more of the first and second vacuum drainage bottles 110a, 110b to the vacuum of the third vacuum drainage bottle 110c. For instance, when the third valve handle 113c is rotated to the open state, one or more of the first and second vacuum drainage bottles 110a, 110b can be exposed to the vacuum of the third vacuum drainage bottle 110c thereby creating a vacuum in the upper unfilled regions of one or more of the first and second vacuum drainage bottles 110a, 110b, allowing or causing one or more of the first and second vacuum drainage bottles 110a, 110b to draw in additional drainage fluid 102.

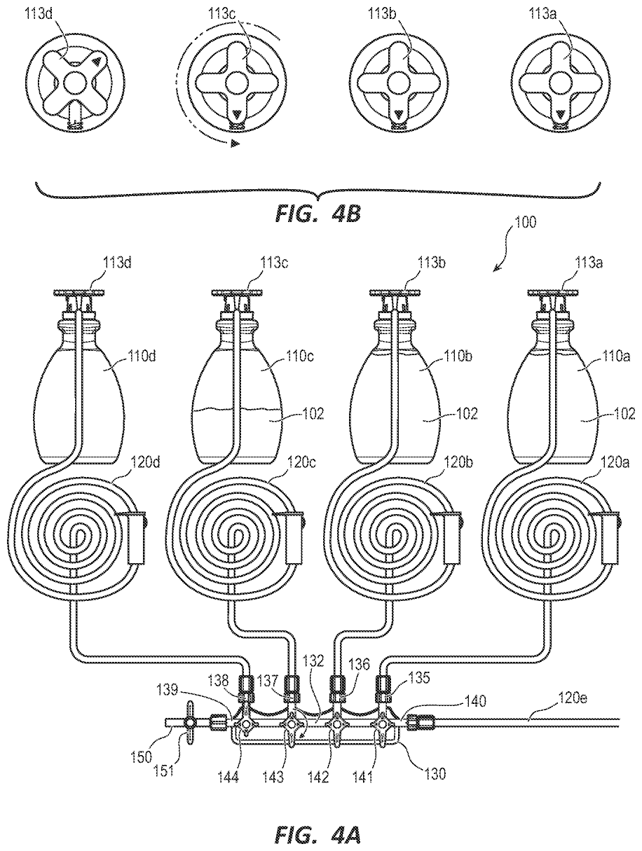

As illustrated in FIGS. 5A and 5B, when the first vacuum drainage bottle 110a, the second vacuum drainage bottle 110b, and the third vacuum drainage bottle 110c are filled with a drainage fluid 102 to desirable levels, the fourth valve handle 113d is rotated to the open state to expose the fourth drainage tube 120d to the vacuum of the fourth vacuum drainage bottle 110d. The fourth rotatable valve 144 is rotated to expose the fluid channel 132 and the fifth drainage tube 120e to the vacuum of the fourth vacuum drainage bottle 110d. The drainage fluid 102 is drawn through the fifth drainage tube 120e, the fluid channel 132, the fourth side port 138, the fourth drainage tube 120d, and into the fourth vacuum drainage bottle 110d. In some embodiments, when the fourth valve handle 113d is rotated to the open state, the drainage fluid 102 level within one or more of the first, second, and third vacuum drainage bottles 110a, 110b, 110c can increase due to the exposure of one or more of the first, second, and third vacuum drainage bottles 110a, 110b, 110c to the vacuum of the fourth vacuum drainage bottle 110d. For instance, when the fourth valve handle 113d is rotated to the open state, one or more of the first, second, and third vacuum drainage bottles 110a, 110b, 110c can be exposed to the vacuum of the fourth vacuum drainage bottle 110d thereby creating a vacuum in the upper unfilled regions of one or more of the first, second, and third vacuum drainage bottles 110a, 110b, 110c, allowing or causing one or more of the first, second, and third vacuum drainage bottles 110a, 110b, 110c to draw in additional drainage fluid 102.

Alternatively, in another embodiment, a plurality or all of the drainage bottles 110a, 110b, 110c, 110d coupled to the multi-part manifold 130 can be used to collect drainage fluid simultaneously (e.g., into the first drainage bottle 110a, into the second drainage bottle 110b, into the third drainage bottle 110c, and into the fourth drainage bottle 110d, etc. simultaneously via use of the multi-port manifold), such as by opening all the rotatable valves 141, 142, 143, 144 simultaneously or by opening the second, third, and fourth rotatable valves 142, 143, 144 prior to opening the first rotatable valve 141 (the valve nearest the patient).

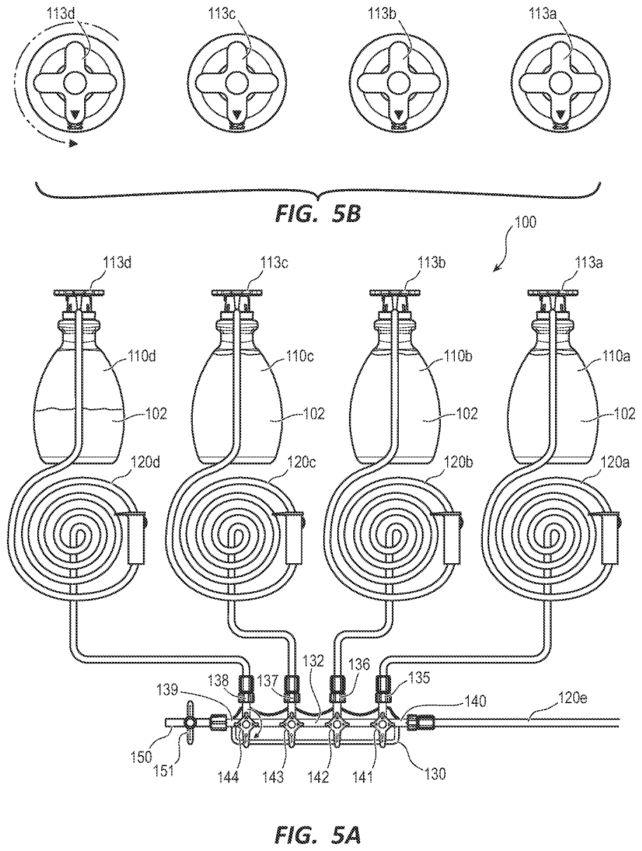

FIGS. 6A and 6B illustrate the vacuum drainage collection system or kit 100 in a drainage sampling state. As illustrated, a drainage fluid sampling device 170 is coupled to the stopcock valve 150. The rotatable handle 151 of the stopcock valve 150 is rotated to an open position such that the drainage fluid 102 is allowed to flow from the fluid channel 132 of the multi-port manifold 130, through the stopcock valve 150, and into the drainage fluid sampling device 170. In some embodiments, the drainage fluid sampling device 170 may be a syringe. In other embodiments, the drainage fluid sampling device 170 may be a vacuum tube.

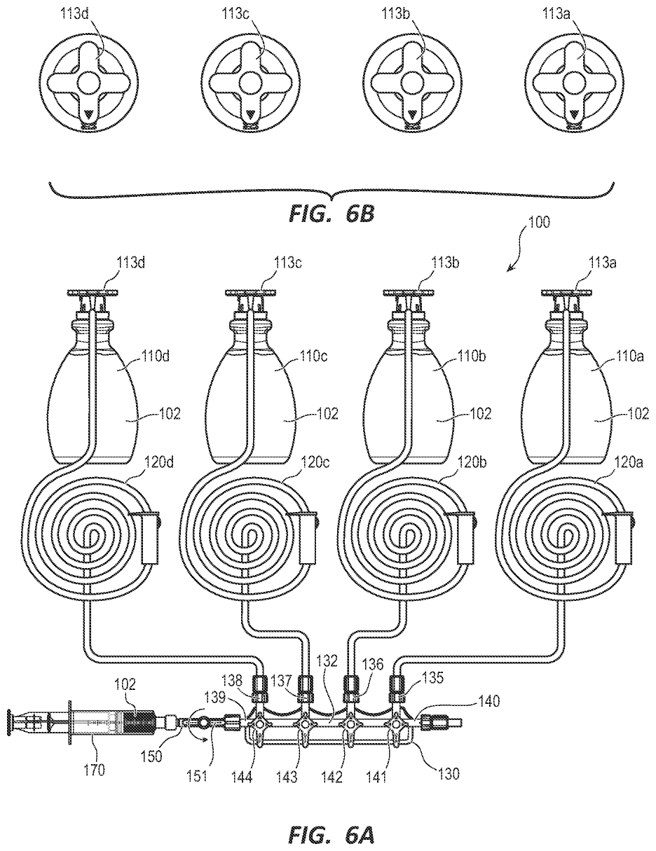

FIG. 7 depicts an embodiment of a vacuum drainage bottle 210 that resembles the vacuum drainage bottle 110 described above in certain respects. Accordingly, like features are designated with like reference numerals, with the leading digit incremented to “2.” For example, the embodiment depicted in FIG. 7 includes a container 212 that may, in some respects, resemble the container 112 of FIG. 1A. Relevant disclosure set forth above regarding similarly identified features thus may not be repeated hereafter. Moreover, specific features of the container 112 and related components shown in FIGS. 1A-6B may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the container 212 and related components depicted in FIG. 7. Any suitable combination of the features, and variations of the same, described with respect to the container 112 and related components illustrated in FIGS. 1A-6B can be employed with the container 212 and related components of FIG. 7, and vice versa. This pattern of disclosure applies equally to further embodiments depicted in subsequent figures and described hereafter, wherein the leading digits may be further incremented.

FIG. 7 illustrates another embodiment of a vacuum drainage bottle 210. In the depicted embodiment, the vacuum drainage bottle 210 includes a cap 211 coupled to a container 212. The container 212 may be formed of a thermochromic material that is configured to change colors in response to a temperature change of the material. In one embodiment, the thermochromic material may be a heat sensitive paint applied to at least a portion of a surface of the container 212. In another embodiment, the thermochromic material may be a heat sensitive pigment added to the material of the container 212. The container 212 can be configured to change colors at a temperature of between about 22 degrees Centigrade (above room temperature) and about 37 degrees Centigrade (approximate temperature of the drainage fluid), or between about 30 degrees Centigrade and about 33 degrees Centigrade. For example, the color of the container 212 below the level of the drainage fluid 202 can be a first color (due to the temperature of the drainage fluid 202) and the color of the container 212 above the level of the drainage fluid 202 can be a second color (due to the absence of the drainage fluid 202 in that region). This configuration results in a transition zone 218 between the first color and the second color that indicates the volume of the drainage fluid 202 within the container 212. As the volume of drainage fluid 202 increases, the transition zone 218 moves upward.

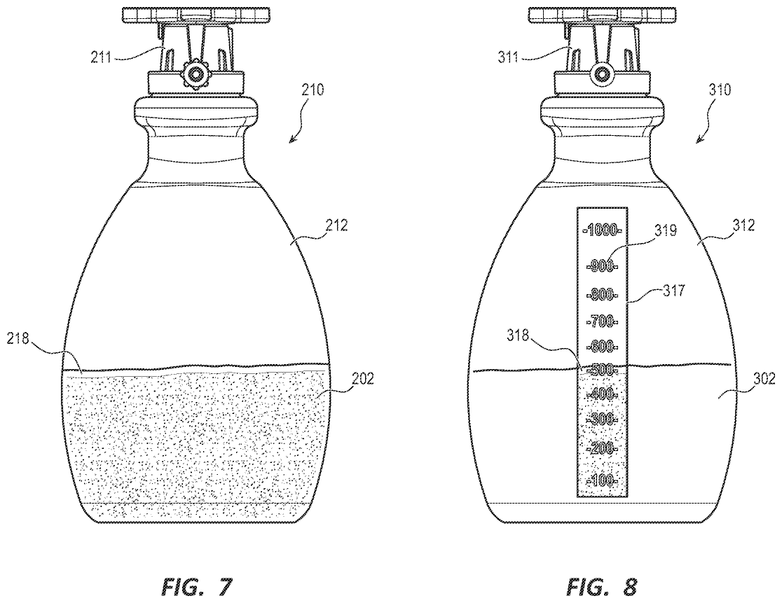

FIG. 8 illustrates another embodiment of a vacuum drainage bottle 310. In the depicted embodiment, the vacuum drainage bottle 310 includes a cap 311 coupled to a container 312. The container 212 may include a label 317 that includes a thermochromic material configured to change colors in response to a temperature change of the thermochromic material. In one embodiment, the thermochromic material may be a thermochromic paint including a heat sensitive pigment that is applied to at least a portion of the label 317. In another embodiment, the thermochromic material may be a heat sensitive pigment added to the material used to form the label 317. The label 317 can be configured to change colors at a temperature of between about 22 degrees Centigrade (above room temperature) and about 37 degrees Centigrade (approximate temperature of the drainage fluid), or between about 30 degrees Centigrade and about 33 degrees Centigrade. For example, the color of the label 317 below the level of the drainage fluid 302 can be a first color (due to the temperature of the drainage fluid 302) and the color of the label 317 above the level of the drainage fluid 302 can be a second color (due to the absence of the drainage fluid 302 in that region). This configuration results in a transition zone 318 between the first color and the second color that indicates the volume of the drainage fluid 302 within the container 312. As the volume increases, the transition zone 318 moves upward.

In certain embodiments, the label 317 may include printed indices 319 to indicate a volumetric measurement of the drainage fluid 302 within the container 312. For example, the printed indices 319 may indicate the volume of the drainage fluid 302 in one or more of milliliters and fluid ounces. In some embodiments, the ink of the indices 319 may include a thermochromic pigment such that when the level of the drainage fluid 302 within the container 312 reaches one indicium of the indices 319, the indicium changes color to indicate the volume of the drainage fluid 302 within the container 312.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of collecting drainage fluid in a vacuum drainage bottle system may include one or more of the following steps: coupling a first drainage tube to a first vacuum drainage bottle and a first port of a manifold; coupling a second drainage tube to a second vacuum drainage bottle and a second port of the manifold; rotating a first valve handle of the first vacuum drainage bottle to open the first vacuum drainage bottle and expose the first drainage tube to a vacuum pressure; rotating a second valve handle of the second vacuum drainage bottle to open the second vacuum drainage bottle and expose the second drainage tube to a vacuum pressure; rotating a second valve coupled to the second port; and rotating a first valve coupled to the first port to allow drainage fluid to flow through the first and second drainage tubes into the first and second vacuum drainage bottles simultaneously. The method of collecting drainage fluid in a vacuum drainage bottle system may further include one or more of the following steps: coupling a third drainage tube to a third vacuum drainage bottle and a third port of the manifold; rotating a third valve handle of the third vacuum drainage bottle to open the third vacuum drainage bottle and expose the third drainage tube to a vacuum pressure; rotating a third valve coupled to the third port to allow drainage fluid to flow through the third drainage tube into the third vacuum drainage bottle; coupling a fourth drainage tube to a fourth vacuum drainage bottle and a fourth port of the manifold; rotating a fourth valve handle to open the fourth vacuum drainage bottle and expose the fourth drainage tube to a vacuum pressure; and rotating a fourth valve coupled to the fourth port to allow drainage fluid to flow through the fourth drainage tube into the fourth vacuum drainage bottle. Other steps are also contemplated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

In the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

“Fluid” is used in its broadest sense, to refer to any fluid, including both liquids and gases as well as solutions, compounds, suspensions, body fluids, etc., which generally behave as fluids.

References to approximations are made throughout this specification, such as by use of the terms “substantially” and “about.” For each such reference, it is to be understood that, in some embodiments, the value, feature, or characteristic may be specified without approximation. For example, where the qualifier “about” is used, the term includes within its scope the qualified words in the absence of their qualifier. For example, where the term “about 37 degrees Centigrade” is recited with respect to a feature, it is understood that in further embodiments, the feature can have a precisely 37 degrees Centigrade configuration.

The terms “a” and “an” can be described as one, but not limited to one. For example, although the disclosure may recite a kit having “a drainage tube,” the disclosure also contemplates that the kit can have two or more drainage tubes.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A vacuum drainage bottle system, comprising:
a first vacuum drainage bottle under vacuum pressure;
a first drainage tube coupled to the first vacuum drainage bottle;
a second vacuum drainage bottle under vacuum pressure;
a second drainage tube coupled to the second vacuum drainage bottle; and
a manifold comprising a flow channel, a first port in selective fluid communication with the flow channel, and a second port in selective fluid communication with the flow channel,
wherein the first drainage tube is coupled to the first port and the second drainage tube is coupled to the second port, and
wherein the first port is selectively openable to the first drainage tube and the second port is selectively openable to the second drainage tube.

2. The vacuum drainage bottle system of claim 1,
wherein the first vacuum drainage bottle under vacuum pressure comprises:
a first container configured to collect drainage fluid from a patient;
a first cap coupled to the first container;
wherein the first cap is configured to selectively open the first container to expose the first drainage tube to a vacuum pressure; and
wherein the second vacuum drainage bottle under vacuum pressure comprises:
a second container configured to collect drainage fluid from the patient;
a second cap coupled to the second container; and
wherein the second cap is configured to selectively open the second container to expose the second drainage tube to a vacuum pressure.

3. The vacuum drainage bottle system of claim 2, further comprising:
a third vacuum drainage bottle under vacuum pressure comprising:
a third container configured to collect drainage fluid from the patient;
a third cap coupled to the third container; and
a third drainage tube coupled to the third cap and in fluid communication with the third container,
wherein the third cap is configured to selectively open the third container to expose the third drainage tube to a vacuum pressure; and
a fourth vacuum drainage bottle under vacuum pressure comprising:
a fourth container configured to collect drainage fluid from the patient;
a fourth cap coupled to the fourth container; and
a fourth drainage tube coupled to the fourth cap and in fluid communication with the fourth container,
wherein the fourth cap is configured to selectively open the fourth container to expose the fourth drainage tube to a vacuum pressure;
wherein the manifold further comprises a third port in selective fluid communication with the flow channel and a fourth port in selective fluid communication with the flow channel,
wherein the third drainage tube is coupled to the third port and the fourth drainage tube is coupled to the fourth port, and
wherein the third port is selectively openable to the third drainage tube and the fourth port is selectively openable to the fourth drainage tube.

4. The vacuum drainage bottle system of claim 3, wherein the manifold further comprises:
a fifth port in fluid communication with the flow channel, and
a sixth port in fluid communication with the flow channel.

5. The vacuum drainage bottle system of claim 2, wherein each of the first and second caps comprise a valve handle configured to be rotated to selectively open each of the first and second containers,
wherein each of the first and second caps is closed when the valve handle is in a first position, and
wherein each of the first and second caps is open when the valve handle is in a second position.

6. The vacuum drainage bottle system of claim 1, wherein one or more of the first or second vacuum drainage bottles comprises a thermochromic material, and
wherein the thermochromic material is configured to indicate a volume of collected drainage fluid within one or more of the first or second vacuum drainage bottles.

7. (canceled)

8. The vacuum drainage bottle system of claim 1, wherein a thermochromic label is coupled to one or more of the first or second vacuum drainage bottles, and
wherein the thermochromic label is configured to indicate a volume of collected drainage fluid within one or more of the first or second vacuum drainage bottles.

9. (canceled)

10. The vacuum drainage bottle system of claim 1, further comprising a stopcock valve couplable to the multi-port manifold.

11. The vacuum drainage bottle system of claim 1, further comprising:
a third drainage tube couplable to another port and to a drainage catheter inserted into a patient.

12. The vacuum drainage bottle system of claim 11, wherein each of the first, second, and third drainage tubes comprise a first male Luer fitting disposed at a first end and a second mail Luer fitting disposed at a second end.

13. The vacuum drainage bottle system of claim 1, wherein one or more of the first drainage tube comprises a one-way valve to prevent backflow of drainage fluid from the first vacuum drainage bottle or the second drainage tube comprises a one-way valve to prevent backflow of drainage fluid from the second vacuum drainage bottle.

14. A vacuum drainage bottle kit comprising:
a multi-port manifold;
a first vacuum drainage tube couplable to the multi-port manifold;
a second vacuum drainage tube couplable to the multi-port manifold;
a third vacuum drainage tube couplable to the multi-port manifold and a drainage catheter inserted into a patient;
a drainage fluid sampling device coupleable to the multi-port manifold; and
an instructions for use (IFU) sheet or directions for accessing instructions on how to use the vacuum drainage bottle kit.

15. The vacuum drainage bottle kit of claim 14, further comprising:
a first vacuum drainage bottle coupleable to the first vacuum drainage tube; and
a second vacuum drainage bottle coupleable to the second vacuum drainage tube.

16. The vacuum drainage bottle kit of claim 14, further comprising a stopcock valve couplable to the multi-port manifold.

17. The vacuum drainage bottle kit of claim 15, further comprising:
a third vacuum drainage bottle;
a fourth vacuum drainage bottle;
a fourth vacuum drainage tube couplable to the multi-port manifold and the third vacuum drainage bottle; and
a fifth vacuum drainage tube couplable to the multi-port manifold and the fourth vacuum drainage bottle.

18-19. (canceled)

20. The vacuum drainage bottle kit of claim 14, wherein one or more of the first vacuum drainage tube comprises a one-way valve to prevent backflow of drainage fluid or the second vacuum drainage tube comprises a one-way valve to prevent backflow of drainage fluid.

21. A method of collecting drainage fluid in a vacuum drainage bottle system, comprising:
coupling a first drainage tube to a first vacuum drainage bottle and a first port of a manifold;
coupling a second drainage tube to a second vacuum drainage bottle and a second port of the manifold;
opening the first vacuum drainage bottle and exposing the first drainage tube to a vacuum pressure;
opening the second vacuum drainage bottle and exposing the second drainage tube to a vacuum pressure;
rotating a first valve coupled to the first port to allow drainage fluid to flow through the first drainage tube into the first vacuum drainage bottle; and
rotating a second valve coupled to the second port to allow drainage fluid to flow through the second drainage tube into the second vacuum drainage bottle.

22. The method of claim 21, comprising:
rotating the second valve coupled to the second port prior to rotating the first valve coupled to the first port such that drainage fluid flows through the first and second drainage tubes and into the first and second vacuum drainage bottles simultaneously.

23. The method of claim 21, further comprising:
coupling a third drainage tube to a third vacuum drainage bottle and a third port of the manifold;
opening the third vacuum drainage bottle and exposing the third drainage tube to a vacuum pressure;
rotating a third valve coupled to the third port to allow drainage fluid to flow through the third drainage tube into the third vacuum drainage bottle;
coupling a fourth drainage tube to a fourth vacuum drainage bottle and a fourth port of the manifold;
opening the fourth vacuum drainage bottle and exposing the fourth drainage tube to a vacuum pressure; and
rotating a fourth valve coupled to the fourth port to allow drainage fluid to flow through the fourth drainage tube into the fourth vacuum drainage bottle.

24-25. (canceled)

26. The method of claim 21, further comprising:
coupling a stopcock valve to the manifold;
coupling a sampling device to the stopcock valve;
opening the stopcock valve; and
filling the sampling device with the drainage fluid.

27-30. (canceled)
